(19) United States
(12) Patent Application Publication
(10) Pub. No.: US 2005/0288602 A1
(43) Pub. Date: Dec. 29, 2005

(54) Device and method for determining respiratory quotient without measuring lung ventilation

(75) Inventors: John Hoppe, Ventura, CA (US); Andrew Huszcza, Ventura, CA (US)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Vacumetrics Inc.

(21) Appl. No.: 11/143,471

(22) Filed: Jun. 3, 2005

Related U.S. Application Data
(60) Provisional application No. 60/576,572, filed on Jun. 4, 2004.

(51) Int. Cl. A61B 5/08

(52) U.S. Cl. 600/532; 600/531

(57) ABSTRACT

A method for the determining the ratio of oxygen consumption to carbon dioxide production, commonly known as the respiratory quotient, in expired breath, and a device to carry out the method without providing volumetric measurements.

### FIELD OF THE INVENTION

The measurement of respiratory quotient is potentially valuable when employed for diagnostic, therapeutic, muscle exercise monitoring, fitness training and glucose metabolism monitoring purposes. This invention relates to a device and method for determining the respiratory quotient in the expired breath of a human or mammal.

### BACKGROUND OF THE INVENTION

Respiratory quotient (RQ) is a measure of oxygen consumption to carbon dioxide production. Mathematically, RQ can be expressed by the following formula:
RQ=VCO₂/VO₂
where VCO₂ is the volume of carbon dioxide in expired air and VO₂ is volume of oxygen consumption.

RQ has been measured by medical professionals as a useful indicator in monitoring nutrition, ventilatory weaning, and in the management of sepsis, trauma, surgery and bums.

Traditionally, a metabolic measurement system is used for the measurement of respiratory quotient. Such systems are also referred to as metabolic carts, cardio-pulmonary exercise systems (CPX), and in Europe, as ergospirometry systems. The use of CPX systems, also known as indirect calorimetry, is a continuous and non-invasive measurement of respiratory gas exchange.

CPX systems have a variety of applications, among them the determination of respiratory quotient for intravenously fed patients in intensive care units. Respiratory quotient is important because it is an indication of proper nutrition, meaning optimal delivery of energy substrates, i.e., carbohydrates and fats infused in the right proportions. Additionally, it has been shown that underfeeding a patient can delay or negatively influence the recovery of a sick patient and manufacturers of metabolic carts try to justify the purchase expense with the resulting savings from wasted overfeeding.

Currently the majority of all hospitals utilize CPX systems, which are relatively expensive, computerized machines, often with questionable accuracy. CPX systems use gas analyzers to determine relative oxygen to carbon dioxide concentration of exhaled breath, and a flow or volume measuring device to measure the volume of exhalation. Therefore, VCO₂ and VO₂ stand for the volume (V) of O₂ and CO₂, conventionally determined for one-minute intervals.

It is therefore an object of the invention to provide an improved method of determination of Respiratory Quotient and a device embodying the method, which is economical.

It is a further object of this invention to provide a device to measure the relative carbon dioxide to oxygen levels of expired breath (with oxygen and carbon dioxide analyzers) without the need to take volumetric measurements.

Further and other objects of the invention will be apparent to one skilled in the art when considering the following summary of the invention and the more detailed description of the preferred embodiments illustrated herein.

### SUMMARY OF THE INVENTION (& ADVANTAGES)

The Haldane Transformation refers to the multiplication of inspired oxygen concentration by the ratio of expired to inspired nitrogen concentrations in the calculation of the inhaled oxygen volume (VIO2) when only the exhaled breath volume is typically known.
The knowledge of the inhaled oxygen volume (VIO2) is needed, as oxygen consumption is the difference between inhaled and exhaled oxygen volumes:
   VO₂═V₁·% O_{2I}−V_{E}·% O_{2E}
Where V_{I} is the inhaled volume, % O_{2I} is the oxygen concentration in inhaled volume, V_{E} is the exhaled volume and % O_{2E} is the oxygen concentration in exhaled volume. Since inert gases, predominantly Nitrogen (N₂) are neither consumed nor produced in the body, their volume remains unchanged:
V_{I}·% N_{2I}═V_{E}·% N_{2E}
where % N₂, and % N_{2E} denote inhaled and exhaled concentrations of Nitrogen. Hence

$${V}_{I} = {V}_{E} * ( \frac{\% {N}_{2 E}}{\% {N}_{2 I}} )$$

can be plugged into the Oxygen consumption equation resulting in:

$${V}_{O2} = {V}_{E} * ( \% {O}_{2 I} * \frac{\% {N}_{2 E}}{\% {N}_{2 I}} - \% {O}_{2 E} )$$

In case of breathing air
% O_{2I}=20.94 and % N_{2I}=79 (78% N₂+1% Argon)
Therefore:
VO₂═V_{E} (% N_{2E}*0.265−% O_{2E})
And, since:
% N_{2E}=100−% O_{2E}−% CO_{2E}
VO₂═V_{E}(26.5−0.265*% O_{2E}−0.265×% CO_{2E}−% O_{2E})
Then:
VO₂═V_{E}(26.5−1.265*% O_{2E}−0.265×% CO_{2E})

For computation of carbon dioxide output the formula is as follows thanks to the negligible content of CO₂ in air:
VCO₂═V_{E}(% CO_{2E}−0.04)

Where % CO_{2E} is the exhaled concentration of carbon dioxide and 0.04 represents trace content (0.04%) of CO₂ in air.

Therefore:

$$RQ = \frac{{CO}_{2} - 0.04}{26.5 - 1.265 {O}_{2} - 0.265 {CO}_{2}}$$

where, for simplicity, CO₂ and O₂ mean percentual content of these gases in exhaled volume, since in the mathematical formula representing the calculation of respiratory quotient, RQ=VCO₂/VO₂, the volume value (V) is both in the numerator and the denominator, the ventilatory (VE) components of V “cancel” if the Haldane transformation equation is duly applied. The practical result of employing this transformation is that the lung ventilation does not need to be measured. However, so far no one has made practical use of this simplification.

The invention is a major simplification of the CPX concept. It employs the simplified computations provided herein to calculate respiratory quotient, without the need to take ventilatory measurements. The application of the Haldane transformation equation confers a major simplification over current meters in that lung ventilation does not need to be measured.

The meters according to the invention are less expensive than meters currently on the market.

According to a primary aspect of the invention there is provided a device for determining the respiratory quotient of a patient's expired breath without providing volumetric measurements comprising, a mixing chamber, an oxygen sensor to continuously determine oxygen partial pressure in the expired breath, a carbon dioxide sensor to continuously determine carbon dioxide partial pressure in the expired breath, a Nafion humidity equalization tube and a mixing chamber. Preferably the device is adapted to receive a power supply, and in one embodiment has a user interface for the operation of input switches and a pump.

According to yet another aspect of the invention there is provided a method of measuring the respiratory quotient of expired breath, for example with the device of above mentioned, wherein mixed exhaled O2 and CO2 concentrations (in %) are measured and implemented to determine RQ according to the following formula:

$$RQ = \frac{CO 2 - {CO}_{2 I}}{26.5 - 1.265 \times O 2 - 0.265 \times CO 2}$$

where CO_{2I} is the carbon dioxide concentration in inhaled air, using automatic barometric pressure compensation of the O₂ and CO₂ signals.

In another embodiment the method does not require automatic barometric pressure compensation.

In yet another embodiment there is provided a method of measuring the respiratory quotient of expired breath, for example with the device above mentioned, wherein mixed exhaled O2 and CO2 concentrations (in %) are measured and implemented to determine RQ according to the following formula:

$$RQ = \frac{CO 2 - {CO}_{2 I}}{26.5 - 1.265 \times O 2 - 0.265 \times CO 2}$$

using automatic barometric pressure compensation of the O₂ and CO₂ signals and using a predetermined mixture of O₂, CO₂ and N₂ as a calibration gas to verify the accuracy of the O₂ and CO₂ analysis as well as the accuracy of the RQ computation.

In another embodiment the respiratory quotient of expired breath, for example with the device above mentioned, wherein mixed exhaled O₂ and CO₂ concentrations (in %) are measured and implemented to determine RQ and that does not require automatic barometric pressure compensation, according to the following formula:

$$RQ = \frac{CO 2 - {CO}_{2 I}}{26.5 - 1.265 \times O 2 - 0.265 \times CO 2}$$

using automatic barometric pressure compensation of the O₂ and CO₂ signals and using a predetermined mixture of O₂, CO₂ and N₂ as a calibration gas to verify the accuracy of the O₂ and CO₂ analysis as well as the accuracy of the RQ computation.

### DETAILED DESCRIPTION OF THE PREFERRED EMBODIMENTS INVENTION

The Following Examples are Provided to Illustrate the Teaching of this Invention. They are not Intended to be Limiting Upon the General Scope Thereof.

The major components of the device include oxygen and carbon dioxide sensor, a mixing chamber to mix the exhaled breaths, a “Nafion” humidity equalization tube, a gas pumping device, a subject's airway interfacing gear and a power supply.

The device may have a user interface or display device such as a small color display in order for the user to input commands in the “checklist” style, wherein various variable may be set, in turn, upon prompt, and to give color feedback of the power-up procedure. The interface may have a keyboard for the entry of record data, such as a patient identification number. Optionally, said display may also have a clock for display of time and date, and an alarm to alert the operator of when a low calibration gas level is approaching. Further the device may be adapted to accommodate an external or built-in printer for the output of relevant data to be reviewed by the health professionals.

The panel control components of the user interface may include the following input switches (buttons): an on and off “toggling” switch; a “zeroing” input for the measure of carbon dioxide, an O₂ calibration input to set the correct reading (gain) of oxygen in air, both supplying air to CO₂ and O₂ analyzers; a calibration switch admitting calibration gas to CO₂ and O₂ analyzers and activating means to adjust CO₂ signal gain to obtain required RQ reading and a RUN (or OPERATE) switch admitting mixing chamber's mixed exhaled gases to CO₂ and O₂ analyzer during RQ monitoring operation.

Calibration gas is assumed to be a mixture that results in an RQ value of 0.90 s, such as 16.6% O₂, 4% CO₂, balance N₂; an RQ value of 0.77, such as 16% O₂, 4% CO₂, balance N₂ or any other target RQ

In a typical operation, the user would first turn the machine on. The device might then perform a self-check.

Operation:

1. Turn power ON

2. Leave for at least 30 minutes for warm-up in RUN mode.

3. Switch to O₂ CAL and adjust reading to 20.94

4. Switch to O₂ ZERO and adjust to within −0.01 to +0.01

5. Turn the CAL GAS “ON” and switch to RQ CAL. Wait at least 30 seconds and then adjust reading to a target RQ value, typically 0.90

6. Turn off calibration gas and switch to operation mode RUN.

7. The apparatus is ready to monitor human or experimental animal RQ.

1. A device for determining the respiratory quotient of a human or mammal's expired breath without providing volumetric measurements comprising, a mixing chamber, an oxygen sensor to continuously determine oxygen concentration in the expired breath, a carbon dioxide sensor to continuously determine carbon dioxide concentration in the expired breath, a Nafion humidity equalization tube, a gas pumping device and a subject's airway interfacing gear.

2. The device according to claim 1 which is adapted to receive a power supply.

3. A device according to claim 1, which has a user interface for the operation of input switches and a pump.

4. A method of measuring the respiratory quotient of expired breath with the device of claim 1 wherein mixed exhaled O2 and CO2 concentrations (in %) are measured and implemented to determine RQ according to the following formula:
$$RQ = \frac{CO 2 - CO 2 I}{26.5 - 1.265 O 2 - 0.265 CO 2}$$
using automatic barometric pressure compensation of the O2 and CO2 signals.

5. A method according to claim 4 that does not require automatic barometric pressure compensation.

6. A method of measuring the respiratory quotient of expired breath with the device of claim 1 wherein mixed exhaled O2 and CO2 concentrations (in %) are measured and implemented to determine RQ according to the following formula:
$$RQ = \frac{CO 2 - CO 2 I}{26.5 - 1.265 O 2 - 0.265 CO 2}$$
using automatic barometric pressure compensation of the O2 and CO2 signals and using a predetermined mixture of O2, CO2 and N2 as a calibration gas to verify the accuracy of the O2 and CO2 analysis as well as the accuracy of the RQ computation.

7. A method of measuring the respiratory quotient of expired breath with the device of claim 1 wherein mixed exhaled O2 and CO2 concentrations (in %) are measured and implemented to determine RQ and that does not require automatic barometric pressure compensation, according to the following formula:
$$RQ = \frac{CO 2 - CO 2 I}{26.5 - 1.265 O 2 - 0.265 CO 2}$$
using a predetermined mixture of O2, CO2 and N2 as a calibration gas to verify the accuracy of the O2 and CO2 analysis as well as the accuracy of the RQ computation.
